(19) United States
(12) Patent Application Publication
(10) Pub. No.: US 2026/0157654 A1
(43) Pub. Date: Jun. 11, 2026

(54) Characterizing Anatomical Height of a Longitudinal Arch of a Foot, and a Single Index Therefore

(71) Applicant: Ian Kenton Engelman, Portland, ME (US)

(72) Inventor: Ian Kenton Engelman, Portland, ME (US)

(21) Appl. No.: 18/975,147

(22) Filed: Dec. 10, 2024

(51) Int. Cl. A61B 5/107; A61B 5/00

(52) U.S. Cl. CPC A61B 5/1074; A61B 5/0064

(57) ABSTRACT

A method characterization of a longitudinal foot arch height by a single index comprises placing the forefoot in plantigrade posture and preforming 3D scan thereon, to obtain a 3D map of the foot, and identifying a line extending between the fifth metatarsal and a point under the geometrical bottom center of the plantar surface under the heel, and measuring the maximal height of the plantar surface from the line, to obtain the height of the arch as a parameter of the index. Optionally the index has additional parameters.

### FIELD OF THE INVENTION

The present invention relates generally to methods of characterizing human feet, and more particularly to a method for characterizing anatomical height of a longitudinal arch of a foot, utilizing a single index.

### BACKGROUND OF THE INVENTION

The human foot is a complex mechanical structure designed to support various movements and bear the body's weight. Due to the stress imposed on the foot and due to numerous pathologies, various types of foot orthoses are often prescribed by foot health practitioners such as orthotists, and simple orthoses commonly known as insoles or Orthotics are commonly used with and without prescription for increased comfort and support. The shape, type, and dimensions of such orthosis is preferably decided based on the foot shape and dimensions of the individual user. Since the foot geometry does not follow common geometrical shapes such as cubes and cylinders, and varies from one individual to the next, and since the measurement technique changes with every clinician, such characterization is inconsistent.

A common activity practitioners execute involves measuring the part of the foot colloquially known as the longitudinal arch of the foot. The longitudinal arch extends about the longitudinal middle of the foot, is highest on the medial (internal) side, and slopes down towards the lateral (external) side of the foot. This foot arch area is not traditionally characterized numerically. Instead an impression of the lower foot is acquired. Furthermore, when studying generalities of foot anatomy and the best parameters for designing foot orthosis, it is desirable that the number of parameters be minimized, as such parametrization will greatly simplify research and setting recommendations for orthosis type and structure in accordance to common diagnoses and geometries.

Three dimensional (3D hereinafter) scanning is a well known technique for generating a digital representation of an image scene including depth information. The digital representation is known as a 3D map or 3D image. A common 3D scanning device is embedded in certain mobile telephone sets. Certain models of iPhone (Apple Corp, Cupertino, California USA) include a 3D scanning features called TrueDepth 3D which shall be used by way of example in the present disclosure. It is stressed that other 3D scanning devices may be used provided they meet the desired accuracy.

Presently, a primary method of orthotic geometry collection is by having a patient provide an impression of the foot pressed into a soft material that maintains its shape after the impression is made, and the impression is then used to construct a cast or a 3D map of the foot bottom. This method is colloquially called a crush box impression. Another traditional method is by taking a plaster or fiberglass cast of the foot. Yet another method involves 3D scanning of a foot, however the scanning results are inconsistent because of foot position inconsistencies, soft tissue compression inconsistencies, and weight bearing inconsistencies.

A shortfall of the crush box impression technique, casting technique as well as the present 3D scanning techniques stems from the numerous ways that practitioners may capture the foot shape. The variety of ways to take the impression or the scan the foot causes different results between practitioners as well as, sometimes, between successive measurements of the same foot whether done by one or multiple practitioners. By way of example a measurement will change by adding dorsiflexion on the first phalange, adducting the forefoot, partial weight bearing, full weight bearing, siting, and standing, varying foot angles, and the like.

The various errors resulting from the great variations in obtaining consistent foot geometry presents significant problems to orthotics manufacturers, and causes a large number of patients to receive an orthotic which does not provide the desired comfort and/or therapeutic effect. Without a repeatable methodology of arch height measurement, the goal of predictably providing well fitting orthotic becomes elusive.

Having a single parameter, or more precisely a small grouping of parameters, to characterize the arch would greatly simplify research and general characterization of the foot, even at the cost of some minor inaccuracies of individual feet. Such a parameter or group of parameters acts as an index, providing unique characterization of the foot geometry or some critical portions thereof.

It is seen therefore that there is a clear, yet heretofore unmet need for a method for characterization of a foot arch, by uniform measurement of the foot arch height.

### SUMMARY OF THE INVENTION

An aspect of the present relates to consistent identifying of a foot arch height of a patient utilizing a single index, namely the arch height index, which may be named equivalently merely as the index. The index comprises at least an arch height measurement, and optionally a location of the arch height maxima along an index line extending at least between two reference points on the plantar surface of the foot

Other parameters may also be present in the index. As stated above, consistent characterization of the foot arch by such single index would greatly assist the state of the art, even at a cost of some loss of completeness. Therefore, a basic goal of this disclosure is directed to obtaining such arch height index. For brevity, this disclosure designates a point C on the plantar surface, about the center of the geometrical bottom of the heel, a point M1 approximating the geometrical center of the plantar surface under the first metatarsal head, and a point M5 approximating the geometrical center of the plantar surface under the fifth metatarsal head. An optional additional point is point M4 which approximates the geometrical center of the plantar surface under the fourth metatarsal head.

The foot is often described as being in three parts, the hindfoot, midfoot, and the forefoot. The hindfoot consists of the talus and calcaneus. The midfoot consists of the five tarsal bones: navicular, cuboid and three cuneiforms. The forefoot consists of the metatarsals sesamoids, and phalanges. Primarily, while standing on a flat surface, the contact points to the foot from the ground are the hindfoot and the forefoot. The hindfoot contacts the ground at the calcaneus, and the forefoot contacts the ground at the metatarsal heads (and sesamoids), shaft of the fifth metatarsal, and base of the fifth metatarsal.

The posture of the foot will be different if there is proximally directed and applied force to both the hindfoot and the forefoot, rather than just the forefoot. Proximally directed force to the hindfoot will cause the calcaneus to plantarflex. This will materially affect the contours of the plantar surface of the foot and will decrease the height of the longitudinal arch of the foot.

The term plantar surface should be construed as relating to any part of the skin of the lower surface of the foot.

Placing the forefoot in a known posture is advantageous for obtaining a consistent and representative measurement of the arch height index. Such posture is named a plantigrade posture hereinafter. To aid in defining a plantigrade posture, these specification will utilize a plane that will be referred to as a tibial-centric plane.

FIGS. 1-2 depict various views of the tibial-centric plane and the plantigrade posture, and the description of respective figures further elaborate those features to ease understanding of placement of the foot in plantigrade posture.

FIGS. 1A-1C depict skeletal views of a lower leg 800 and are provided to aid in visualizing the tibia centric plane 810. FIG. 1A depicts a coronal plane view of the lower leg, FIG. 1B is a sagittal plane view of the lower leg, and FIG. 1C is a transverse plane view of the foot. Line 820 depicts the tibia center line, which is a line extending from approximately the center of the tibia plateau to approximately the center of the tibial talar articulation surface, or stated differently, approximately along the longitudinal axis of the tibia 830. The tibia center line 820 lies in the tibial-centric plane 810 and is one parameter defining the plane. The tibial-centric plane is further defined by being parallel to a metatarsal line 410 which extends between points M1 and M5, or alternatively M4. Notably, the tibial-centric plane is defined referencing the respective forefoot and the shaft of the tibia.

The term plantigrade posture as defined in these specification, denotes any forefoot posture at which a line, named herein a metatarsal line, extending between the first and fifth metatarsal heads, and being projected onto the tibial-centric plane, will be perpendicular to the tibia center line or any extension thereof. Alternatively the metatarsal line may extend between the first and fourth metatarsal head.

FIG. 2A depict schematically an anterior coronal plane view and FIG. 2B an oblique view, showing skeletal elements of a forefoot in an exemplary plantigrade posture in accordance with the definition provided above. The metatarsal line 410 is shown as 410′ when projected onto the plane 810. A forefoot or foot is said to be in plantigrade posture when the line 410′ is substantially perpendicular to the tibia centerline 820 as shown schematically by angle 910. Notably, the foot may be in plantarflexion or dorsiflexion and still be in plantigrade posture. It is again noted that the tibia centerline 820 extends indefinitely and the projected line 410′ may lie at, above, or below the tibial talar articulation surface.

If during measuring of the arch height index the underfoot tissue is compressed, the results are likely to be adversely effected. Therefore, for accurate and consistent measurements the underfoot tissue should be substantially free of tissue compression at least at points M1 and C and along an index line extending therebetween. Thus, for the purposes of this specification, the term plantigrade posture also implies substantially the lack of compressive forces on the lower foot tissue as described above. A single index may include a single parameter or a small parameter set. By way of non limiting example an Arch Height Index (or equivalently ‘index’ hereinafter) may comprise a single parameter expressing height H, of the arch above a certain plane or comprise two parameters {H, D}, such as the height H and a distance D along the index line from an agreed upon or stated base point to the location corresponding with H, or comprise three parameters {H, L, D}, where the L parameter representing a total length of the index line 100 between given base points. Thus, by way of example if the index line 100 extends between selected base points such as C and M1, then in a three parameter index, the length of the index line 100 is the optional length parameter L of the single index, the distance from one of points C or M1 to the point below the highest point of the arch is the distance parameter D of the index, and the maximal height H of the arch plantar surface from the index line 100 along the line, is the height parameter. Thus the optional three parameter index may be in the form {Height, Length, Distance} but a single parameter index may be {H}, a two parameters index may be {H, L} or {H, D} and the like. An index may also be expressed with missing parameter, such as {H,, D}, which may be used to indicate a missing length parameter. Additional parameters may be added to the index by convention. The distance parameter D may be expressed as an absolute measurement or as a relative value such as, by way of example, 35%, which implies the distance at which the maximal arch height was measured is at 35% of the line length L measured from conventionally agreed upon base point such as M1. Similarly, the height parameter may be expressed as a ratio between the index line length L and the the measured height.

For brevity, except when a specific bone or bone structure is specified, such as the tibia by way of example, when a point is specified on the foot or the forefoot, it should be understood that such point relate to a point which is on the plantar surface, or stated differently on the skin of the lower foot, indicative of the anatomical structure above the point. Similarly, a measurement performed between a line, a plane or the like and the foot, the measurement extends between the respective line or plane and the like and plantar surface at the specified direction, if applicable. Thus, by way of example, the points M1 and M5 describe points on the plantar surface under the first and fifth metatarsal heads, while the tibia center line 820 relates to the center line of the tibia bone 830 itself.

To resolve the shortcomings described above, there is provided a method of measuring an arch height index of a foot having an arch, a heel, a plantar surface, a forefoot, and first and fifth metatarsal heads, the arch height index comprising at least an anatomical arch height parameter, the method comprising:
a) placing the foot at plantigrade posture;
b) preforming a 3D scan of the patient foot while in plantigrade posture, to obtain a 3D map;
c) Utilizing the 3D map, estimating a point C residing on the plantar surface and being indicative of the geometrical bottom center of the heel, estimating a point M1 residing on the plantar surface under the first metatarsal head, and estimating a point M5 residing on the plantar surface under the fifth metatarsal head;
d) In the 3D map, identifying a base plane having therein points C, M1 and M5;
e) utilizing the 3D map, performing a plurality of height measurements extending between the plane and the plantar surface of the foot, the height measurements being performed along an index line extending between point C and point M1, each measurements being performed orthogonally to the plane, to obtain a height measurements set;
f) determining the anatomical arch height parameter by locating a maxima in the height measurements set.

In certain embodiments the method described above may be performed as two separate methods, the first method comprising steps a. and b above, while the second method comprises steps d)-e), and optionally step f). The first and second methods may be performed by differing entities, such as by way of example, a foot treatment practitioner may perform the steps of placing the foot at plantigrade posture and performing the scan of the foot in that orientation, yet the resulting 3D map is being utilized by a second entity such as an orthotist which performs the identification of points M1, M5, and C, performing the measurements and determining the index parameters. Thes step f) may be performed by either of the first and second entity or by a third entity. The steps d)-f) may be performed manually or by a computer.

Optionally, the arch height index further comprise a distance parameter the distance parameter corresponding to distance along the index line extending between M1 and C where the maxima of the arch height parameter resides. Such distance parameter is obtained by determining the distance along the index line from point M1 or C to a point where the maxima was obtained. The distance parameter may be expressed by any desired measurement unit, such as length, ratio, or percent of the index line. Further optionally, the arch height index may comprise the length of the index line. Notably, the distance measurement may be performed from any desired base point along the index line, such as an intersection of the line and another line extending at any desired angle from the longitudinal extreme of the foot, and the like As long as such base point is defined, either by convention or by indication within the index, such measurement would serve similar purposes. Stated differently, while the index line contains points C and M1, it may extend in any direction, and any point along this line may serve as a base point for distance parameter D, as selected by convention.

Optionally the 3D scan is performed utilizing a cellular telephone or a tablet.

In one embodiment the 3D scan is performed utilizing a device having a camera supporting TrueDepthtm technology or a similar depth-sensing technology. TrueDepthtm is a depth-sensing technology trademark, (owned by Apple Inc, Cupertino, California) In another exemplary embodiments, the 3D scan is performed utilizing laser triangulation technology, structured light technology, stereoscopic camera, time-of-flight laser scanning, conoscopic holography technology, modulated light technology, photometric technology and the like. Combination of 3D scanning technologies may also be utilized.

### BRIEF DESCRIPTION OF THE DRAWINGS

The summary above, and the following detailed description will be better understood in view of the enclosed drawings which depict details of preferred embodiments. It should however be noted that the invention is not limited to the precise arrangement shown in the drawings and that the drawings are provided merely as examples.

FIGS. 1A, 1B, and 1C depict schematically skeletal lower leg in coronal plane view, sagittal plane view and transverse plane view respectively.

FIG. 2A depict schematically a skeletal anterior view of a lower leg, and FIG. 2B an oblique view, showing the forefoot in plantigrade posture.

FIG. 3 depicts a bottom view of a foot showing geometry for obtaining a single height index characterizing foot arch height.

FIG. 4 depicts a side view of a foot, depicting schematically various parameter of the arch height index.

FIG. 5 depict a foot in the process of the foot being measured.

FIG. 6A depicts an example of a 3D map, and FIG. 6B depicts a simplification of a 3D map showing foot depth contours.

FIG. 7 depicts a simplified process flow.

FIG. 8 depicts a bottom view of a foot showing a plurality of optional base points for index line

### DETAILED DESCRIPTION

The ensuing description, together with the accompanying figures, makes apparent to a person having ordinary skill in the pertinent art how the teachings of the disclosure may be practiced, by way of non-limiting examples. The figures are for the purpose of illustrative discussion and no attempt is made to show structural details of an embodiment in more detail than is necessary for a fundamental understanding of the disclosure. For the sake of clarity and simplicity, some objects depicted in the figures may not be drawn to scale.

While some of the figures depict a left foot and some a right foot, it will be clear that corresponding contralateral feet are merely mirror images of the depicted foot figures, and the invention scope extends equally to right and left foot.

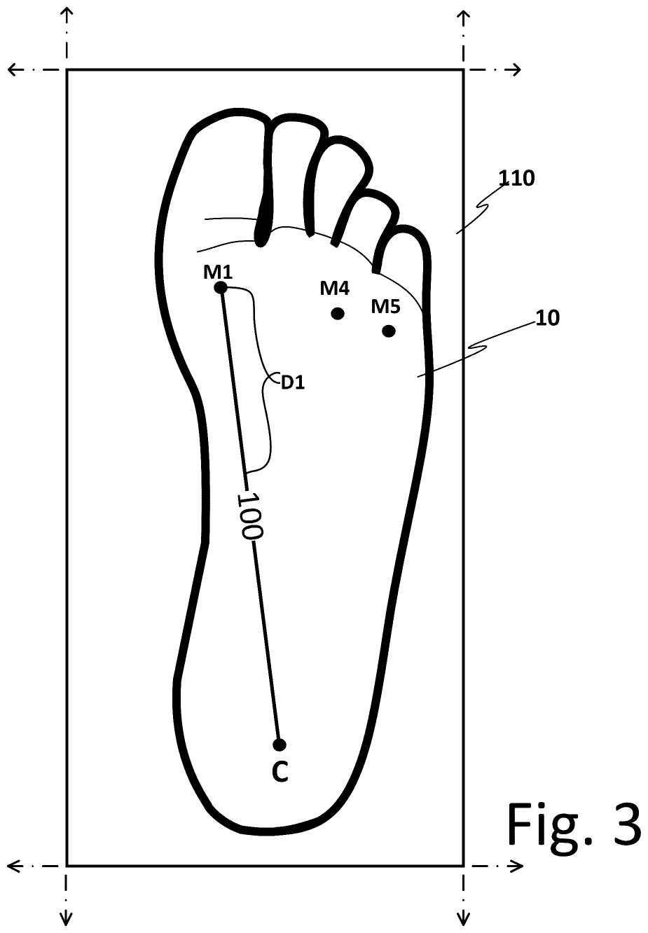

FIG. 3 depicts a bottom view of a foot 10, denoting three points on the plantar surface, namely the approximate center of foot heel-identified as point C, the approximate center of the first metatarsal-identified as point M1, and the approximate center of the fifth metatarsal, identified as point M5. As previously stated, the above identified three points are located on the foot skin under their respective anatomical structure, or stated differently, on the plantar surface of the relevant foot. By way of example point M1 may be referred to as the ‘first metatarsal point’ and such reference should be construed as relating to the plantar surface under the first metatarsal.

The three points C, M1, and M5 define a base plane 110, which contains the three points. FIG. 3 further depicts a point M4 residing under the fourth metatarsal head, which may be equivalently utilized instead of M5, as such change would have little if any effect on the measured arch height index

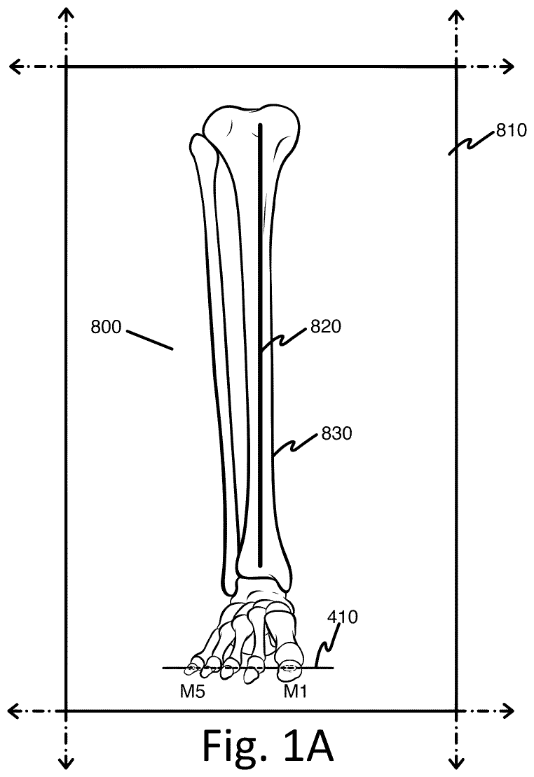

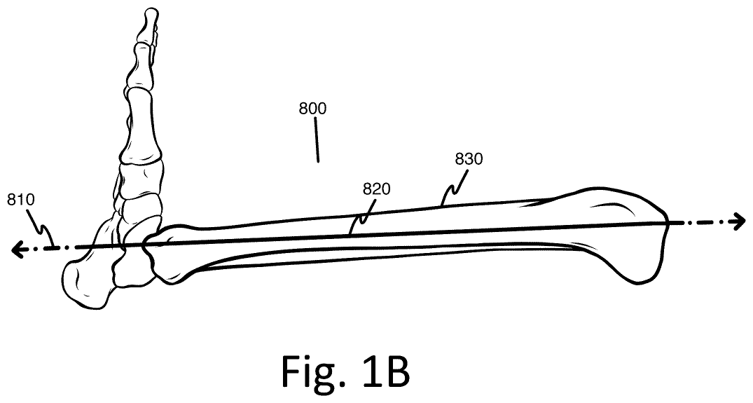

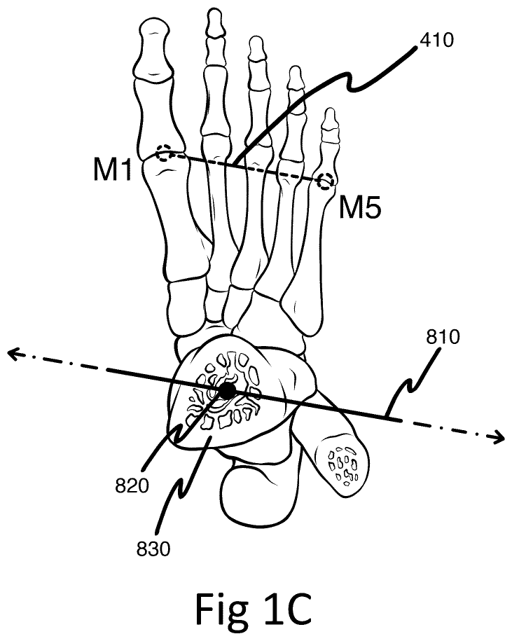

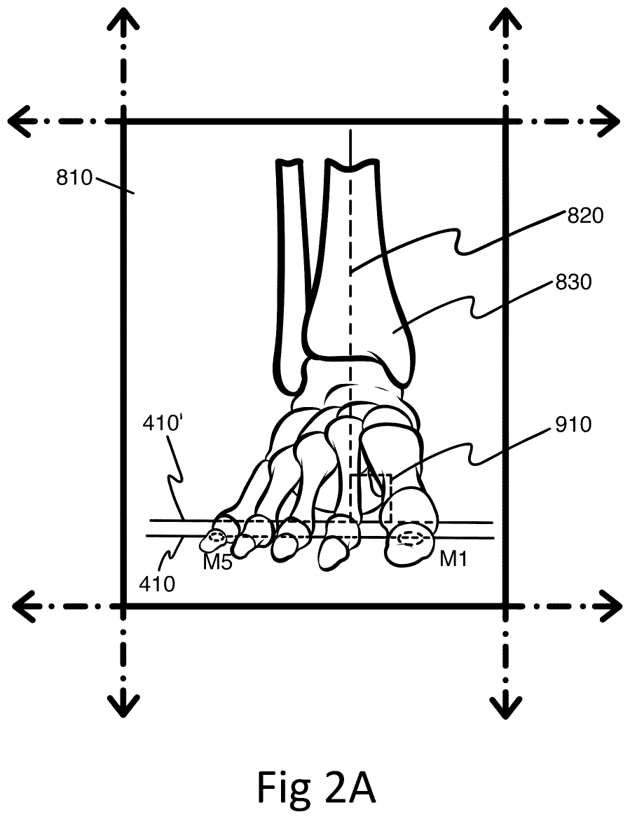

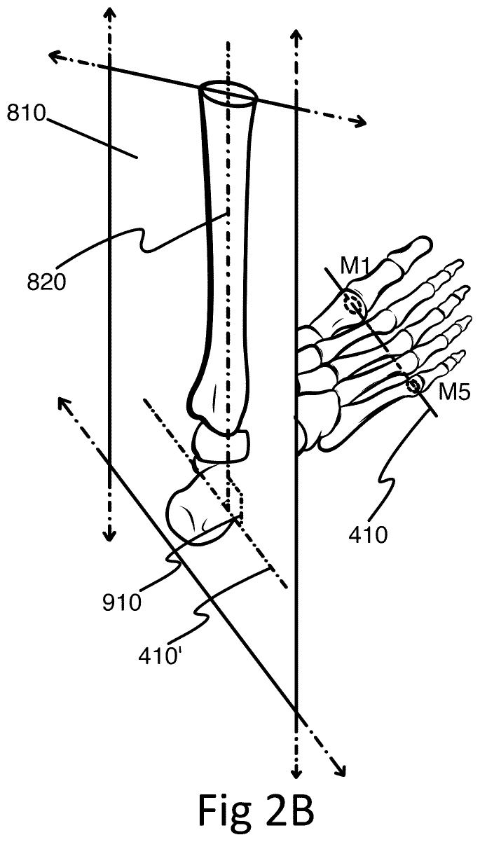

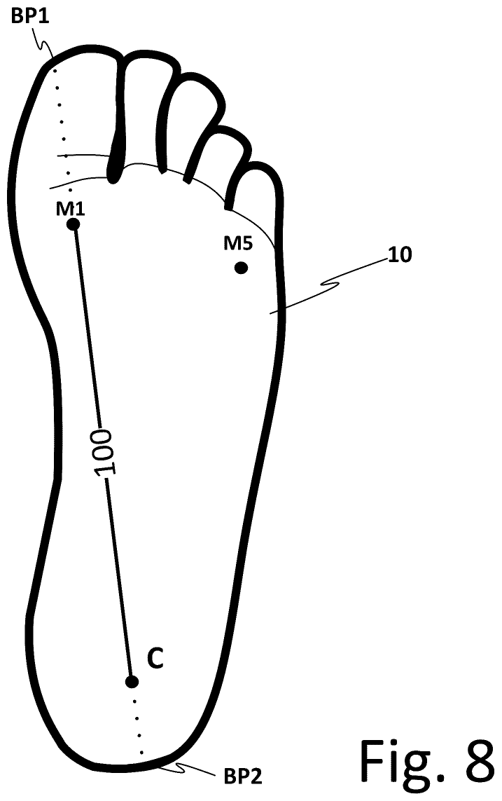

FIG. 3 further depicts an index line 100 extending between points C and M1. If utilized, the distance parameter D of the arch height index is represented in FIG. 1 by bracket D1, which depicts the distance between reference point M1 and the point where the height parameter H is located along the index line 100. Notably, the distance may be expressed from either point M1 or point C, or from any other selected base point serving as a reference. By way of example, FIG. 8 represent but two optional base points, namely BP1 which is selected at the point where index line 100 meets the distal termination of the foot, and BP2 which is selected at the point index line 100 intersects with the proximal end of the foot. Base points are points along the index line 100 and may be selected by any desired feature at any desired location. However the skilled in the art would recognize that in order to provide universal consistency of the index, such base point is preferably represented consistently between various measurements. Furthermore, the distance may be expressed by actual distance units or as a percentage or ratio between selected base points, such as, by way of example, between points M1 and C, an arbitrary base point such as BP1 and C, or BP2 and M1, and the like.

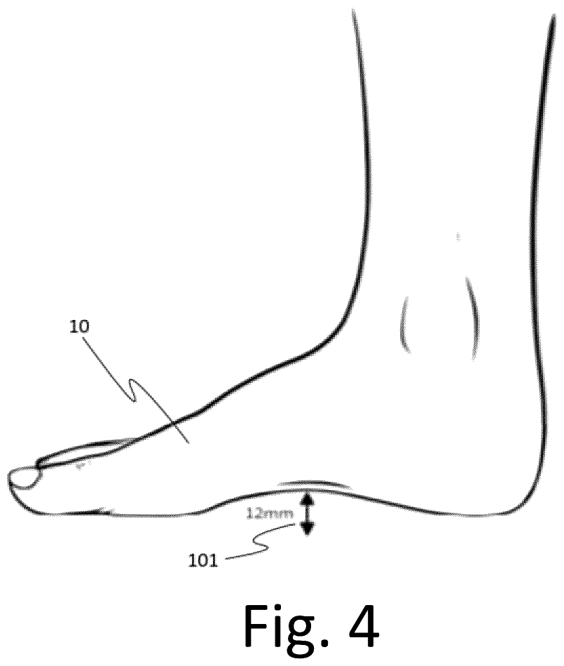

FIG. 4 depicts a side view of a foot 10, depicting schematically the height parameter H of the arch height index, depicted as 101 as being 12 mm by way of example.

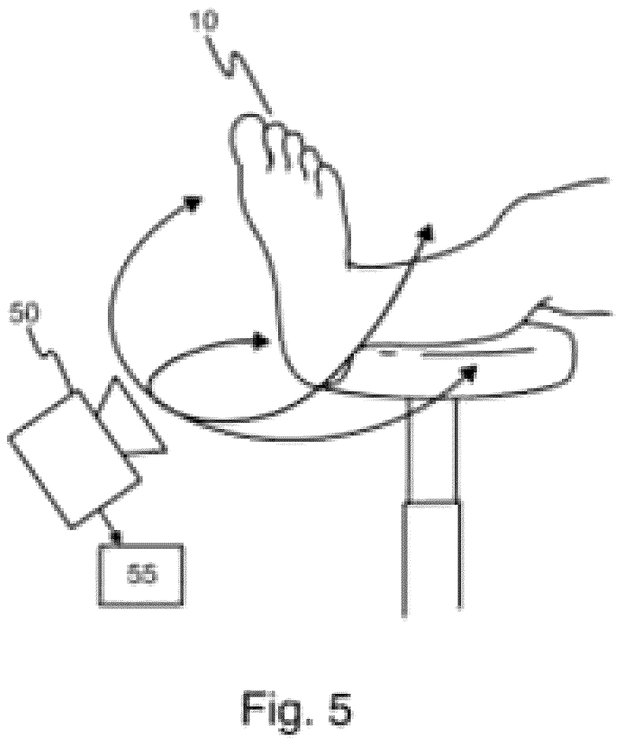

FIG. 5 depicts schematically a foot 10 being scanned. A 3D scanner 50 is deployed to perform the three dimensional scan of the foot, and provide three dimensional map 55 thereof. In certain embodiments a cellular telephone device or a tablet is used as the three dimensional scanner 50, and the arced arrows represent schematically moving the device about the foot, either randomly or in predetermined manner. Notably certain scanner 50 may remain stationary and movement of the camera in any manner depends on the scanner 50 type. The scanner 50 may be of any type, including a mobile device such as a cellular telephone, a tablet, a camera, a lidar, a dedicated scanner and the like. The scanner 50 may comprise a plurality of devices, either similar or dissimilar.

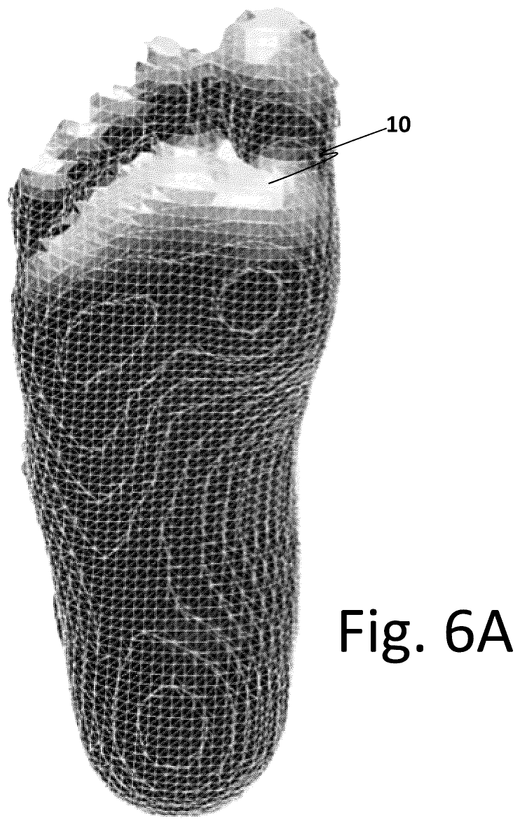

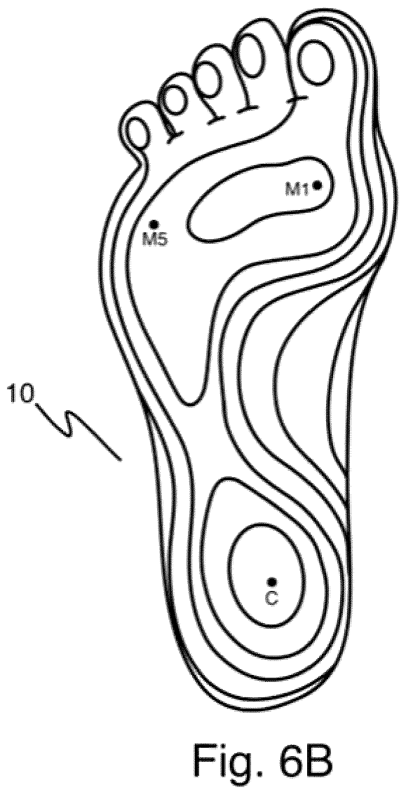

FIG. 6A depicts an example of an actual 3D map 55 of a foot. Depth contours are noticeable in the image, and such contours may be added by software. FIG. 6B depicts a simplification of a 3D map showing the foot depth contours, with points M1, M5 and C superimposed thereupon. Points C, M1 and/or M5 may be determined manually, or automatically by such computerized techniques as height measurements, identifying the contours of the fat pad under the heel and deriving a geometrical center thereof, and the like. It is noted that while the contours simplify identifying the point C, and optionally a more precise location of points M1 and M5, contours are not required and are thus optional.

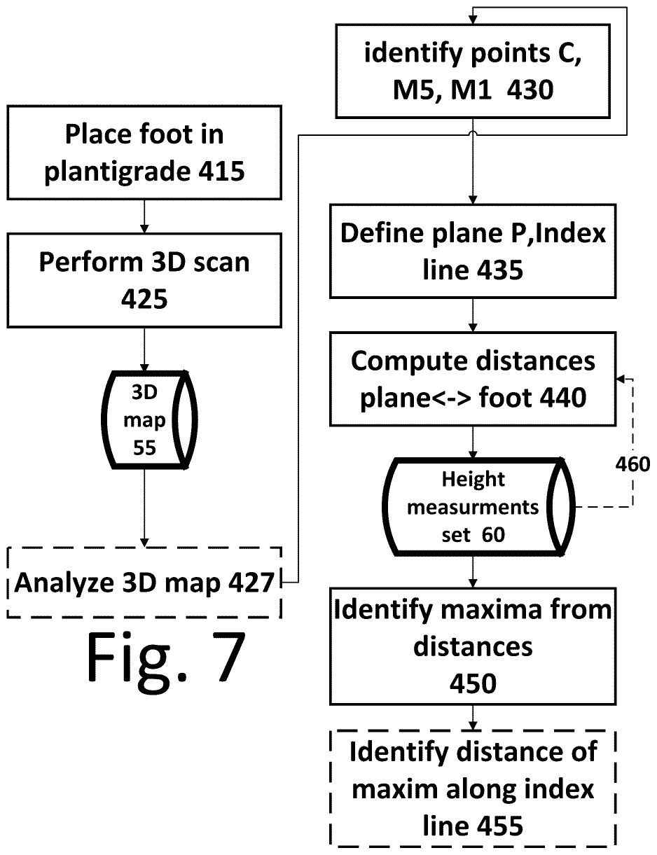

FIG. 7 depicts a simplified flow diagram of a process to identify a height of the foot 10 arch.

The foot is placed substantially in plantigrade posture 415. While a precise disposition of the foot is desired, absolute precision is not required and an approximation is sufficient.

Once the foot 10 is substantially in the plantigrade posture, the foot is scanned 425 by a 3D scanner 50. The 3D scan results in a 3D map 55. The 3D map is a conceptual map, which may be expressed in numerous ways. In its simplest form, the 3D map comprises a plurality of captured points having coordinates in a common coordinate space. The 3D map may also be modified to a form which eases defining the scanned geometry, such as the graphical presentation shown by way example in FIGS. 6 and 6A. The 3D map may be expressed by numerous other data abstractions and/or data structures, such as contours, Bezier curves, and the like. The 3D map may optionally be compressed.

Optionally the 3D scan is analyzed to provide depth contours therein 427.

Points C, M1 and M5 (or M4) are then identified 430 in the 3D map 55. Alternatively, the points are manually identified.

As mentioned above point M4, (not shown) may be identified as equivalent or in addition to point to point M5.

Points C and M1 define an index line 100 and points M1 and M5 define a metatarsal line 410 in the 3D map, as shown by way of example in FIG. 1. Index line 100 and metatarsal line 410 (or points which reside in those lines) define 435 a plane 110. Notably line 100 and/or 410 may extend to any base point but are defined by containing their respective points. Optionally point M4 (shown in FIG. 3), which approximates within the 3D map the geometrical center of the plantar surface covering the fourth metatarsal head, may be equivalently utilized instead of M5, as doing so will have minimal, if any, effect on the accuracy of the arch height index measurement.

Utilizing the 3D map, with the index line 100 and plane 110, the 3D map 55 is utilized to compute 440 a plurality of height distances, which are measured orthogonally to the plane 110 along the index line 100, each measurement expresses the distance between the plane 110 to the foot skin above the respective point in index line 100 at the place where the measurement is carried out. The distances are measured orthogonally to plane 110. The plurality of the measured height distances form a set {M} 60 of height measurements of the foot skin above the plane 110 along index line 100. The step size along index line 100 between individual measurements forming the set {M} 60 is selected in accordance with a desired precision level, and such selection may be fixed by convention or may be varied by a user. By way of example, a precision level of 1 mm may be set as step size along index line 100 between successive height measurements in set {M}. The height measurements obtained in 440 are repeated 460 at each successive step size along at least a portion of the line 100. The skilled in the art would easily recognized numerous methods of defining the number of height measurements performed, such as, by way of example, starting at a selected point of index line 100 such as point M1, and repeatedly performing the height measurements at successive step size distances until point C. An alternative method again start at a selected point along 100, preform the measurements at each step size until a height measurement is greater than zero by a selected amount, and measure successively at step size until the measured height drops below the selected amount or to zero.

The arch height index parameter H is determined by identifying 450 the maxima in the set of height measurements {M}. While in certain embodiments the arch height index comprises only the arch height parameter H, the maxima location along the index line 100 may also be obtained 455, and the distance between the base point and the maxima point comprises the distance parameter D of the arch height index if such parameter is utilized. The length of the index line 100 provides yet another optional parameter of the arch height index, namely the length parameter L.

As mentioned above the process may be split several ways. While the placing of the foot in plantigrade posture 415 and scanning the foot 425 may be carried out by a single entity, additional steps may be carried out by other entity or entities. By way of example the 3D map may be transferred to a second entity which performs at least steps 427,430, 435, and the loop 460 to compute the heights of the foot skin from the plane 440 so as to receive the height measurement set 60. Finding the index parameter H by identifying the maxima 450 of set 60 may be conducted by a third entity. Other divisions of the tasks may occur without detracting from the invention and the claims should be construed to cover any division of the tasks between various entities.

To the extent necessary to understand or complete the disclosure of the present invention, all publications, patents, and patent applications mentioned herein, including in particular the applications of the Applicant, are expressly incorporated by reference in their entirety by reference as if fully set forth herein.

Unless otherwise specified, relational terms used in these specifications should be construed to include certain tolerances that the skilled in the art would recognize as providing equivalent functionality. By way of example the term perpendicular is not necessarily limited to exactly 90.0°, but also to any slight variation thereof that would provide substantially equivalent functionality for the purposes described for the relevant member or element, and/or would not detrimentally affect the measured arch height index. Adjectives such as “about” and “substantially” that modify a condition or relationship characteristic of a feature or features of an embodiment of the present technology, are to be understood to mean that the condition or characteristic is defined to within tolerances that are acceptable for operation of the embodiment for an application for which it is intended, or within variations expected from the measurement being performed and/or from the measuring instrument being used, or sufficiently close to the location, disposition, or configuration of the relevant element to preserve operability of the element within the invention which does not materially modifies the invention. Similarly, unless specifically specified or clear from its context, numerical values should be construed to include certain tolerances that the skilled in the art would recognize as having negligible importance as it does not materially change the operability of the invention. When the term “about” precedes a numerical value, it is intended to indicate +/−15%, or +/−10%, or even only +/−5%, and in some instances the precise value. It is further noted that whenever a foot is said to be placed in plantigrade posture, such placement is done within acceptable tolerances, as precise disposition is not required and an approximation is sufficient. Therefore while a precise plantigrade posture is desired, as defined above when the metatarsal line 410 is projected as 410′ onto the tibial-centric plane 810 and 410′ is perpendicular to the tibia center line 820, slight variations from such perpendicular relationship are not only allowed and still fall between within the plantigrade posture, but such small variations are indeed to be expected, as the positioning of the foot in plantigrade orientation is commonly done by estimation. A skilled foot health practitioner would readily recognize a substantially plantigrade posture and will be able to direct a patient to sufficiently correct posture by pulling more or less on the lanyard so as to allow measuring of the arch height index within acceptable tolerances. Notably, whenever the term ‘and/or’ is used in these specifications and the attached claims, it should be construed as any number, combination or permutation of all, one, some, a plurality or none of each of the item or list mentioned. It is also understood that “(s)” appended to the end of a word designates either singular or plural of the word. It is further understood that “or” is an inclusive “or” to include all items in a list and not intended to be limiting and means any number, combination or permutation of all, one or plurality of each of the item or list mentioned, unless the term ‘or’ is explicitly defined as exclusive, or if the context would clearly indicate an exclusive or to the skilled artisan. In the description and claims of the present disclosure, each of the verbs, “comprise” “include” and “have”, and conjugates thereof, are used to indicate that the object or objects of the verb are not necessarily a complete listing of features, members, components, elements, steps or parts of the subject or subjects of the verb.

In this specification reference is often made to the accompanying drawings which form a part of the disclosure, and in which are shown by way of illustration and not of limitation, exemplary implementations and embodiments. Further, it should be noted that while the description provides various exemplary embodiments, as described and as illustrated in the drawings, this disclosure is not limited to the implementations described and illustrated herein, but can extend to other embodiments as would be known or as would become known to those skilled in the art. Reference in the specification to “one embodiment”, “this embodiment”, “these embodiments”, “several embodiments”, “selected embodiments”, “some embodiments” or conjugates thereof means that a particular feature, structure, or characteristic described in connection with the relevant embodiment(s) may be included in one or more implementations and/or embodiments, and the appearances of these phrases in various places in the specification are not necessarily all referring to the same embodiment(s). Additionally, in the description, numerous specific details are set forth in order to provide a thorough disclosure, guidance and/or to facilitate understanding of the invention or features thereof. However, it will be apparent to one of ordinary skill in the art that these specific details may not all be needed in each implementation. In certain embodiments, well-known structures and materials have not been described in detail, and/or may be illustrated schematically or in block diagram form, so as to not unnecessarily obscure the disclosure. Stated differently, features described as applying to one embodiment may be incorporated with other embodiment(s), and all described features are required for any or all of the disclosed embodiments.

For clarity the directional terms such as ‘medial’, ‘lateral’, ‘anterior’, ‘posterior’, ‘proximal’, ‘distal’, ‘inferior’, ‘superior’, ‘up’, ‘down’, ‘left’, ‘right’, and descriptive terms such as ‘upper’ and ‘lower’, ‘above’, ‘below’, ‘sideways’, ‘inward’, ‘outward’, and the like, are applied according to their ordinary and customary meaning, to describe relative disposition, locations, and orientations of various components. When relating to the drawings, such directional and descriptive terms and words relate to the drawings to which reference is made. Notably, the relative positions are descriptive and relative to the above described orientation such as an orientation which would be exercised during upright walking and/or standing and modifying the orientation would not change the disclosed relative structure. Positional or motional terms such as “upper”, “lower”, “right”, “left”, “bottom”, “below”, “lowered”, “low”, “top”, “above”, “elevated”, “high”, “vertical”, “horizontal”, “front”, “back”, “backward”, “forward”, “upstream” and “downstream”, as well as grammatical variations thereof, may be used herein for exemplary purposes only, to illustrate the relative positioning, placement or displacement of certain components, to indicate a first and a second component in present illustrations or to do both. Such terms do not necessarily indicate that, for example, a “bottom” component is below a “top” component, as such directions, components or both may be flipped, rotated, moved in space, placed in a diagonal orientation or position, placed horizontally or vertically, or similarly modified.

Although the foregoing invention has been described in detail by way of illustration and example, it will be understood that the present invention is not limited to the particular embodiments, options, alternatives, and examples provided in the description and the drawings. Specific embodiments described but may comprise any combination of the above disclosed elements and their equivalents and variations thereof, as well as those combinations, changes and/or modifications which will be obvious to those skilled in the art in view of the present disclosure. The invention extends to such variations and modifications as fall within the true spirit and scope of the invention.

1. A method of measuring an arch height index of a foot having a longitudinal arch, a heel and first and fifth metatarsal heads, the arch height index comprising at least an anatomical arch height parameter {H}, the method comprising:
a) preforming a three dimensional (3D) scan of the patient foot while in plantigrade posture, to obtain a 3D map;
b) in the 3D map, estimating a point C, residing on a plantar surface under a geometrical bottom center of the heel, estimating a point M1 residing on the plantar surface under the first metatarsal head, and estimating a point M5 residing on the plantar surface under the fifth metatarsal head;
c) identifying a base plane having therein points C, M1 and M5;
d) utilizing the 3D map, performing a plurality of height measurements extending between the base plane and a scanned skin of the foot, each measurements being performed orthogonally to the base plane and toward the foot, and is geometrically incident to a segment of an index line, the segment extending between point C and point M1, the plurality of height measurements forming a height measurements set {M}; and,
e) determining the anatomical arch height parameter {H} by locating a maxima in the height measurements set {M}.

2. (canceled)

3. The method as claimed in claim 1, wherein the arch height index further comprises a distance parameter {D}, and wherein the method further comprising determining the parameter {D} by measuring a distance along the index line between a base point and a point under the anatomical arch height parameter {H}.

4. The method as claimed in claim 3, wherein the parameter {D} is represented as a fraction or percentage of a length of the index line.

5. The method as claimed in claim 1, wherein the arch height index further comprises a length parameter {L}, and wherein the method further comprising measuring a length of the index line to obtain the parameter {L}.

6. The method of claim 1 wherein at least a portion of the step of performing the 3D scan is performed at least in part, utilizing a mobile telephony device, a tablet, or a computer.

7. The method of claim 1 wherein at least a portion of the step of performing the 3D scan is performed utilizing a device having a camera system supporting depth-sensing technology.

8. The method of claim 1 wherein at least a portion of the step of performing the 3D scan is performed utilizing at least one of laser triangulation technology, structured light technology, stereoscopic camera, time-of-flight laser scanning, conoscopic holography technology, modulated light technology, or any combination thereof.

9. A method of measuring an arch height index of a foot having a longitudinal arch, a heel and first and fifth metatarsal heads, the arch height index comprising at least an anatomical arch height parameter {H}, the method comprising:
a) obtaining a three dimensional (3D) map of the foot in a plantigrade posture;
b) in the 3D map, estimating a point c, residing on a plantar surface under a geometrical bottom center of the heel, estimating a point M1 residing on the plantar surface under the first metatarsal head, and estimating a point M5 residing on the plantar surface under the fifth metatarsal head;
c) identifying a base plane having therein points C, M1 and M5;
d) utilizing the 3D map, performing a plurality of height measurements extending between the base plane and a scanned skin of the foot, each height measurement being performed orthogonally to the base plane toward the foot, and geometrically incident to a segment of an index line, the segment containing point C and point M1, the plurality of height measurements forming a height measurements set {M}; and,
e) determining the anatomical arch height parameter {H} by locating a maxima in the height measurements set {M}.

10. (canceled)

11. The method as claimed in claim 9, wherein the arch height index further comprises a distance parameter {D}, and wherein the method further comprises determining the parameter {D} by measuring a distance along the index line between a base point and a point under the anatomical arch height parameter {H}.

12. The method as claimed in claim 9, wherein the arch height index further comprises a length parameter {L}, and wherein the method further comprising measuring a length of the index line to obtain the parameter {L}.

13. The method as claimed in claim 9, wherein the arch height index further comprises a distance parameter {D} and a length parameter {L}, and wherein the method further comprises:
determining the parameter {D} by measuring a distance along the index line between a base point and a point under the anatomical arch height parameter {H}; and,
measuring a length of the index line to obtain the parameter {L}.

14. The method as claimed in claim 1, wherein the arch height index further comprises a distance parameter {D} and a length parameter {L}, and wherein the method further comprises:
determining the parameter {D} by measuring a distance along the index line between a base point and a point under the anatomical arch height parameter {H}; and,
measuring a length of the index line to obtain the parameter {L}.
